(19) United States
(12) Patent Application Publication
(10) Pub. No.: US 2009/0053110 A1
(43) Pub. Date: Feb. 26, 2009

(54) MULTIFUNCTIONAL EARPHONE AND ALCOHOL MONITORING SYSTEM USING THE SAME

(75) Inventors: CHIH-WEI CHANG, Jen Te (TW); CHIN-YUAN CHEN, Jen Te (TW)

Correspondence Address: PCE INDUSTRY, INC.;ATT. CHENG-JU CHIANG, 458 E. LAMBERT ROAD, FULLERTON, CA, 92835, US

(73) Assignee: CHI MEI COMMUNICATION SYSTEMS, INC., Tainan County (TW)

(21) Appl. No.: 11/933,991

(22) Filed: Nov. 1, 2007

(30) Foreign Application Priority Data
Aug. 20, 2007 (CN) 200710201407.0

(51) Int. Cl. G01N 1/22; G01N 33/497

(52) U.S. Cl. 422/84; 73/23.3

(57) ABSTRACT

A multifunctional earphone includes an earphone body, and an alcohol-testing module provided in the earphone body. The alcohol-testing module includes an alcohol-testing unit provided with an alcohol concentration-measuring program to measure an alcohol concentration of a user of the multifunctional earphone to obtain a measured alcohol concentration value; and a communication unit to receive the measured alcohol concentration value from the alcohol-testing unit, and analyze the measured alcohol concentration value.

### BACKGROUND OF THE INVENTION

1. Field of the Invention

Aspects of the invention relate to an earphone, and more particularly to a multifunctional earphone and an alcohol monitoring system using the same.

2. Description of Related Art

Motor vehicle crashes caused by alcohol-impaired drivers are a significant social and economic problem. A driver who has been drinking may hardly notice that his/her driving ability has been impaired by the alcohol at the time, but the slow reaction time that can result from even a slight case of alcohol impairment can increase the chances of having an accident, and thus could prove fatal in an emergency driving situation.

If a driver could check the alcohol concentration in his/her body prior to operating a motor vehicle instead of simply relying on how he/she feels to determine whether his/her driving ability has been impaired by the alcohol, he/she might have second thoughts about driving after drinking, thereby reducing the chances of having an accident.

There are several ways to determine the alcohol concentration in a person's body. The alcohol concentration can be roughly estimated using a mathematical approach. One of the most effective ways of doing this is to perform a blood or urine test. However, in some situations, it is inconvenient to perform the blood or urine test. Inexpensive alcohol concentration measuring devices such as a Breathalyzer are available as a simpler and less invasive alternative to the blood or urine test.

However, a Breathalyzer is not considered a device that is widely used and carried in people's daily life. Portable electronic device such as mobile phone on the other hand, are considered a widely used electronic device that is often carried by users at all times. An earphone is often used with such a device to carry on a phone conversation or listen to music.

However, a user who wants to use both a Breathalyzer and an earphone must carry both of them with him at all times, which is inconvenient for the user.

Therefore, a new multifunctional earphone having the function of the Breathalyzer and an alcohol monitor system using the same are desirable to overcome the above-described problems.

### SUMMARY OF THE INVENTION

Several aspects and example embodiments of the invention relate to a new multifunctional earphone and an alcohol monitor system using the same that advantageously enable a user to conveniently perform an alcohol concentration measurement.

In accordance with an example embodiment of the invention, a multifunctional earphone includes an earphone body, and an alcohol-testing module provided in the earphone body. The alcohol-testing module includes an alcohol-testing unit provided with an alcohol concentration-measuring program to measure an alcohol concentration of a user of the multifunctional earphone to obtain a measured alcohol concentration value; and a communication unit to receive the measured alcohol concentration value from the alcohol-testing unit, and analyze the measured alcohol concentration value.

In accordance with another example embodiment of the invention, an alcohol monitor system includes a multifunctional earphone, and an alcohol monitor host. The multifunctional earphone includes an earphone body, and an alcohol-testing module provided in the earphone body to measure an alcohol concentration of a user of the multifunctional earphone to obtain a measured alcohol concentration value and transmit the measured alcohol concentration value. The alcohol monitor host includes a transceiver module to communicate with the alcohol-testing module of the multifunctional earphone and receive the measured alcohol concentration value transmitted from the multifunctional earphone.

In accordance with yet another example embodiment of the invention, a method of measuring an alcohol concentration of a user of a multifunctional earphone includes measuring the alcohol concentration of the user with an alcohol concentration-measuring program provided in the multifunctional earphone to obtain a measured alcohol concentration value; and analyzing the measured alcohol concentration value in the multifunctional earphone.

Additional aspects and/or advantages of the invention will be set forth in the description that follows and, in part, will be obvious from the description, or may be learned by practice of the invention.

### BRIEF DESCRIPTION OF THE DRAWINGS

A better understanding of the invention will become apparent from the following detailed description of example embodiments and the claims when read in connection with the accompanying drawings, all forming a part of the disclosure of the invention. While the following written and illustrated disclosure focuses on disclosing example embodiments of the invention, it should be clearly understood that the same is by way of illustration and example only, and that the invention is not limited thereto. The spirit and scope of the invention are limited only by the terms of the appended claims. The following represents a brief description of the drawings, wherein:

FIG. 1 is a block diagram of an alcohol monitor system according to an example embodiment of the invention;

FIG. 2 is a block diagram of a Bluetooth alcohol-testing module of a multifunctional earphone of the alcohol monitor system shown in FIG. 1; and

FIGS. 3A and 3B are flow charts of operations of the alcohol monitor system shown in FIG. 1.

### DETAILED DESCRIPTION OF THE EMBODIMENTS

Reference will now be made in detail to embodiments of the invention, examples of which are shown in the accompanying drawings, wherein like reference numerals refer to like elements throughout. The embodiments are described below to explain the invention by referring to the figures.

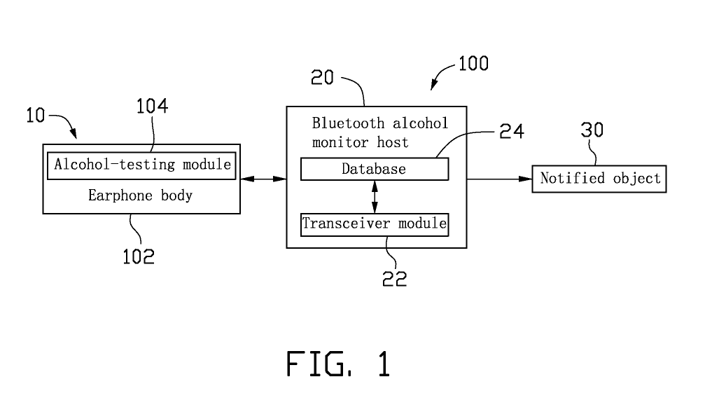

Referring to FIG. 1, an alcohol monitor system 100 includes a multifunctional earphone 10 and a Bluetooth alcohol monitor host 20. The multifunctional earphone 10 is a Bluetooth earphone including an earphone body 102 and a Bluetooth alcohol-testing module 104 provided in the earphone body 102. The Bluetooth alcohol-testing module 104 measures an alcohol concentration in a body of a user of the multifunctional earphone 10, and displays a measured alcohol concentration value as a test result, and/or sends the test result to the Bluetooth alcohol monitor host 20. The Bluetooth alcohol monitor host 20 is a portable electronic device having a communication function, such as a Bluetooth communication function. The portable electronic device may be any portable electronic device with a communication function, such as a mobile phone, a personal digital assistant (PDA), a video camera, a notebook computer, and so on. The Bluetooth alcohol monitor host 20 transmits data to and receives data from the Bluetooth alcohol-testing module 104, and transmits information to a notified object 30. Information enabling the Bluetooth alcohol monitor host 20 to establish a communication link with the notified object 30 is stored in the Bluetooth alcohol monitor host 20. The notified object 30 may be any electronic device having a communication function, such as a mobile communication terminal owned by a family member or a friend of the user of the multifunctional earphone 10, an ignition interlock having a communication function provided in a motor vehicle of the user of the multifunctional earphone 10, or a monitoring device having a communication function operated by a government or private agency that is monitoring the user's compliance with a court-ordered requirement that the user of the multifunctional earphone 10 abstain from using alcohol.

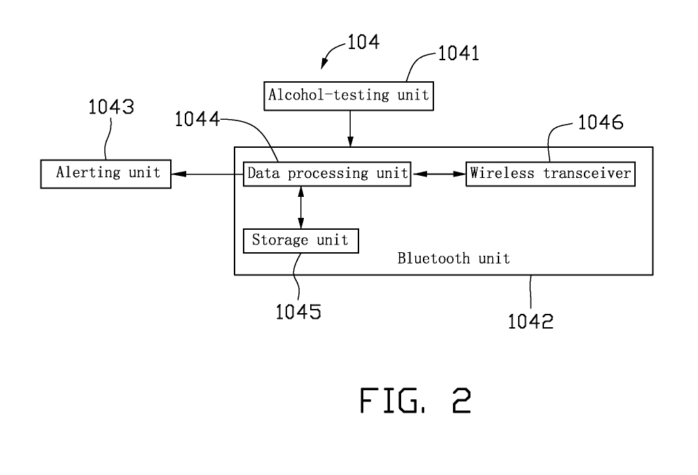

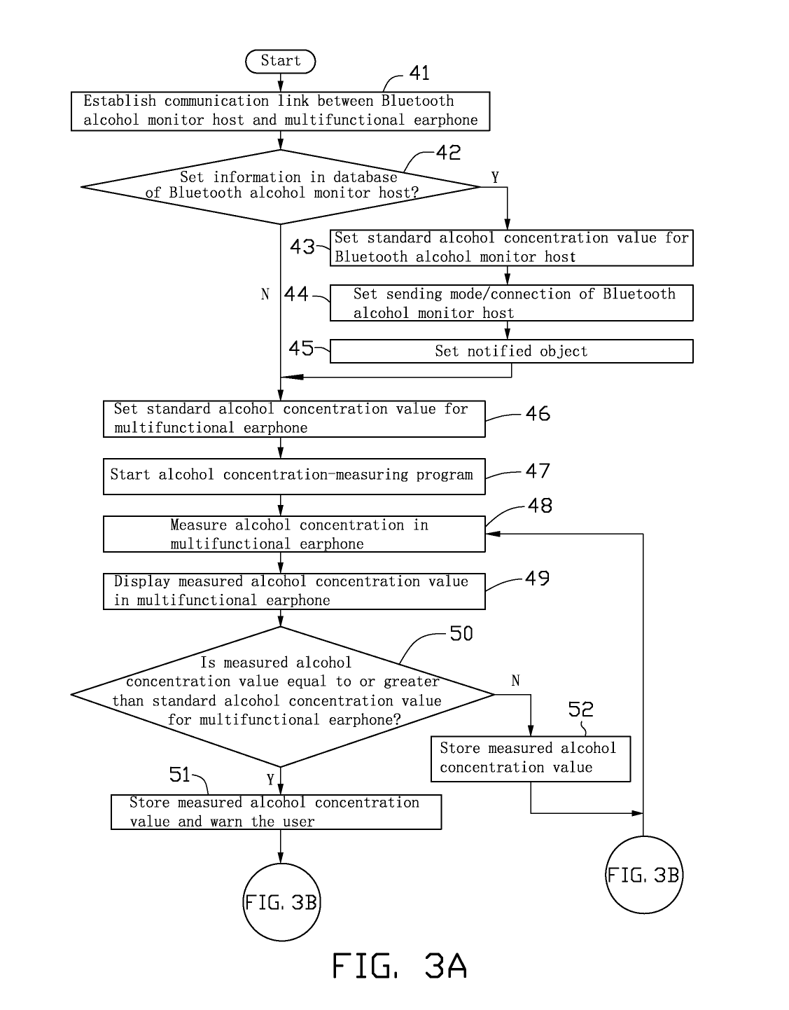

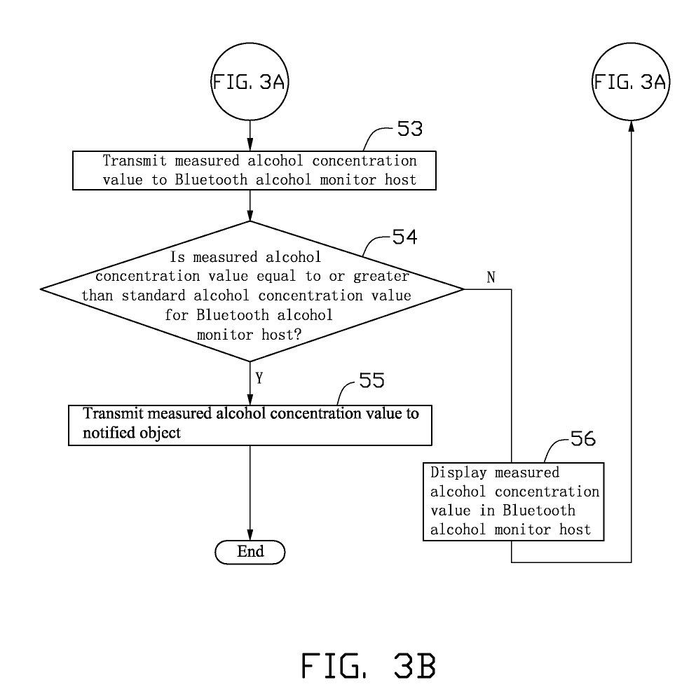

Referring to FIG. 2, the Bluetooth alcohol-testing module 104 includes an alcohol-testing unit 1041 electronically connected to a Bluetooth unit 1042 which is electronically connected to an alerting unit 1043. The alcohol-testing unit 1041 is provided with an alcohol concentration-measuring program for measuring an alcohol concentration in the user's breath and outputting the measured alcohol concentration value to the Bluetooth unit 1042. The alcohol-testing unit 1041 may use any suitable technology for measuring the alcohol concentration in the user's breath. Examples of such technologies are infrared spectrophotometer technology, which is more accurate but also more expensive, and electrochemical fuel cell technology, which is less accurate but also less expensive. Since these technologies are known in the art, they will not be further described herein. The Bluetooth unit 1042 includes a data processing unit 1044, a storage unit 1045, and a wireless transceiver 1046, wherein the data processing unit 1044 is respectively and electronically connected to the storage unit 1045 and the wireless transceiver 1046. The data processing unit 1044 determines whether the measured alcohol concentration value is equal to or greater than a reference alcohol concentration value set for the multifunctional earphone 10. The data processing unit 1044 outputs the measured alcohol concentration value to the storage unit 1045, the alerting unit 1043, and the wireless transceiver 1046 depending on whether the measured alcohol concentration value is equal to or greater than a reference alcohol concentration value set in the multifunctional earphone 10 as described in greater detail below with reference to FIGS. 3A and 3B.

The storage unit 1045 stores the reference alcohol concentration value set for the multifunctional earphone 10 and the measured alcohol concentration value output from the data processing unit 1044. The wireless transceiver 1046 transmits the measured alcohol concentration value to the Bluetooth alcohol monitor host 20. The wireless transceiver may also receive the reference alcohol concentration value set for the multifunctional earphone 10 from the Bluetooth alcohol monitor host 20 as described in greater detail below with reference to FIGS. 3A and 3B. The alerting unit 1043 displays the measured alcohol concentration value and warns the user of the multifunctional earphone 10 that the measured concentration value is equal to or greater than the reference alcohol concentration value set for the multifunctional earphone 10, for example, by beeping and/or vibrating.

Referring again to FIG. 1, the Bluetooth alcohol monitor host 20 includes a transceiver module 22 and a database 24 that are electronically connected together. The transceiver module 22 transmits the measured alcohol concentration value received from the Bluetooth alcohol-testing module 104 to the notified object 30. The measured alcohol concentration value may be transmitted to the notified object 30 through any suitable wireless communication connection, such as a GPRS (general packet radio service) connection, a GSM (global system for mobile communication) connection, a Wi-Fi (wireless fidelity) connection, a CDMA (code division multiple access) connection, a TDMA (time division multiple access) connection, an FDMA (frequency division multiple access) connection, an iDEN (integrated digital enhanced network) connection, a PDC (personal digital cellular) connection, and so on. The database 24 stores information enabling the Bluetooth alcohol monitor host 20 to establish a communication link with the notified object 30, and also stores a reference alcohol concentration value set for the Bluetooth alcohol monitor host 20. As described in greater detail below with reference to FIGS. 3A and 3B, the reference alcohol concentration value set for multifunctional earphone 10 may be less than or equal to the reference alcohol concentration value set for the Bluetooth alcohol monitor host 20. The database 24 is programmed in advance to be able to determine whether the measured alcohol concentration value received by the Bluetooth alcohol monitor host 20 from the Bluetooth alcohol-testing module 104 is equal to or greater than the reference alcohol concentration value set for the Bluetooth alcohol monitor host 20 as described in greater detail below with reference to FIGS. 3A and 3B.

FIGS. 3A and 3B show flow charts of operations of the alcohol monitor system 100 shown in FIG. 1. During use, the multifunctional earphone 10 is located relatively close to a mouth of the user so that it can accurately measure the alcohol concentration in the user's breath. In operation 41, a communication link is established between the Bluetooth alcohol monitor host 20 and the multifunctional earphone 10. In operation 42, the Bluetooth alcohol monitor host 20 determines whether to set the database 24 of the Bluetooth alcohol monitor host 20 based on an input from the user. If the user chooses not to set the database 24, the process proceeds directly to operation 46 (described below). If the user chooses to set the database 24, then in operation 43, a reference alcohol concentration value for the Bluetooth alcohol monitor host 20 is set and stored in the database 24. In operation 44, a sending mode/connection of the Bluetooth alcohol monitor host 20 is set. The sending mode/connection may be GPRS, GSM, Wi-Fi, CDMA, FDMA, iDEN, PDC, and so on. In operation 45, the notified object 30 is set, and the process proceeds to operation 46.

In operation 46, a reference alcohol concentration value for the multifunctional earphone 10 is set and stored in the storage unit 1045. Operation 46 may be performed by the Bluetooth alcohol-testing module 104, or may be performed by the transceiver module 22 of the Bluetooth alcohol monitor host 20 sending an instruction to the Bluetooth alcohol-testing module 104. The reference alcohol concentration value set in the multifunctional earphone 10 may be different from the reference alcohol concentration value set in the Bluetooth alcohol monitor host 20 to meet different requirements. In an example embodiment of the invention, a maximum legally permissible alcohol concentration value is a value equal to A, and the user sets the value A as the reference alcohol concentration value in the Bluetooth alcohol monitor host 20. The user sets another value B slightly less than the value A as the reference alcohol concentration value in the multifunctional earphone 10, so that the user can be notified by the multifunctional earphone 10 before his/her alcohol concentration reaches the maximum legally permissible alcohol concentration value.

The maximum legally permissible alcohol concentration value for a driver of a motor vehicle varies from country to country, and sometimes from area to area within a country. For example, in the U.S.A., by August 2005, all 50 states had passed a law setting the maximum legally permissible blood alcohol content (BAC) for a driver of a motor vehicle at 0.08%. Thus, in the U.S.A., the user might set the reference alcohol concentration value A in the Bluetooth alcohol monitor host 20 at 0.08%, and might set the reference alcohol concentration value B in the multifunctional earphone 10 at 0.05% or 0.06%, for example.

However, it is understood that the user may also set the reference alcohol concentration value B for the multifunctional earphone unit 10 to be equal to the reference alcohol concentration value A for the Bluetooth alcohol monitor host 20.

In operation 47, the alcohol concentration-measuring program provided in the alcohol-testing unit 1041 is started. The starting of the alcohol concentration-measuring program may be performed by the Bluetooth alcohol-testing module 104, or may be performed by the transceiver module 22 of the Bluetooth alcohol monitor host 20 sending an instruction to the Bluetooth alcohol-testing module 104. In operation 48, the multifunctional earphone 10 measures the alcohol concentration in the user's breath. In operation 49, the alcohol-testing unit outputs the measured alcohol concentration value to the Bluetooth unit 1042, and the Bluetooth unit 1042 outputs the measured alcohol concentration value to the alerting unit 1043, which displays the measured alcohol concentration value in the multifunctional earphone 10.

In operation 50, the measured alcohol concentration value received by the Bluetooth unit 1042 is processed by the data processing unit 1044. The data processing unit 1044 determines whether the measured alcohol concentration value is equal to or greater than the reference alcohol concentration value B set for the multifunctional earphone 10 and stored in the storage unit 1045. If the data processing unit 1044 determines that the measured alcohol concentration value is equal to or greater than the reference alcohol concentration value B set for the multifunctional earphone 10 and stored in the storage unit 1045, then operation 51 is performed. In operation 51, the measured alcohol concentration value is stored in the storage unit 1045, and the alerting unit 1043 warns the user that the measured alcohol concentration value is equal to or greater than the reference alcohol concentration value B, for example, by beeping and/or vibrating. The process then proceeds to operation 53. If the data processing unit 1044 determines that the measured alcohol concentration value is less than the reference alcohol concentration value B set for the multifunctional earphone 10 and stored in the storage unit 1045, then operation 52 is activated. In operation 52, the measured alcohol concentration value is stored in the storage unit 1045, and the process returns to operation 48. The Bluetooth alcohol-testing module 104 may be set to repeat operations 48, 49, 50, and 52 at regular intervals so that the alcohol concentration of the user is measured at the regular intervals.

In operation 53, the measured alcohol concentration value stored in the storage unit 1045 in operation 51 is transmitted to the Bluetooth alcohol monitor host 20 by the wireless transceiver 1046 of the Bluetooth alcohol-testing module 104. In operation 54, the database 24 of the Bluetooth alcohol monitor host 20 determines whether the measured alcohol concentration value is equal to or greater than the reference alcohol concentration value A set for the Bluetooth alcohol monitor host 20 in operation 43. If the database 24 determines that the measured alcohol concentration value is larger than the reference alcohol concentration value A set for the Bluetooth alcohol monitor host 20, then operation 55 is performed. In operation 55, the measured alcohol concentration value is transmitted to the notified object 30 by the transceiver module 22. If the database 24 determines that the measured alcohol concentration value is less than the reference alcohol concentration value A set for the Bluetooth alcohol monitor host 20, then operation 56 is performed. In operation 56, the measured alcohol concentration value is displayed in the Bluetooth alcohol monitor host 20, and the process returns to operation 48.

It is understood that numerous modifications of the process shown in FIGS. 3A and 3B are possible, and are encompassed by the invention. For example, in operation 55, the measured alcohol concentration value may also be displayed in the Bluetooth alcohol monitor host 20. Also, operation 56 may be eliminated, and an operation may be provided between operations 53 and 54 to display the measured alcohol concentration value in the Bluetooth alcohol monitor host 20. In such a modification, the process would return to the operation 48 directly from the operation 54 if the database 24 determines that the measured alcohol concentration value is less than the reference alcohol concentration value A set for the Bluetooth alcohol monitor host 20.

It is understood that the multifunctional earphone 10 may also be a wireless or wired earphone that can transmit and receive data without using Bluetooth technology. The Bluetooth unit 1042 may also be a wireless unit or a wired unit that can transmit and receive data without using Bluetooth technology. The alerting unit 1043 may be omitted, and the measured alcohol concentration value may be transmitted to the Bluetooth alcohol monitor host 20 and displayed only in the Bluetooth alcohol monitor host 20. Data may be transmitted and received in the alcohol monitor system 100 using wired connections or wireless connections other than a Bluetooth connection.

One advantage of the multifunctional earphone 10 and the alcohol monitor system 100 using the same is that they enable the user to conveniently perform an alcohol concentration measurement. Additionally, during use, the multifunctional earphone 10 is located relatively close to the mouth of the user so that the alcohol concentration in the user's breath can be accurately measured. Furthermore, the multifunctional earphone 10 integrates a conventional earphone and the Bluetooth alcohol-testing module 104 into a single unit, making it easy for the user to carry both devices at the same time.

While there have been illustrated and described what are considered to be example embodiments of the invention, it will be understood by those skilled in the art and as technology develops that various changes and modifications may be made, and equivalents may be substituted for elements thereof, without departing from the true scope of the invention. Many modifications, permutations, additions, and sub-combinations may be made to adapt the teachings of the invention to a particular situation without departing from the true scope of the invention.

For example, the alcohol-testing unit may use any suitable technology for measuring the alcohol concentration in the user's breath, such as infrared spectrophotometer technology, which is more accurate but also more expensive, and electrochemical fuel cell technology, which is less accurate but also less expensive.

The Bluetooth alcohol monitor host may be any portable electronic device with a communication function, such as a mobile phone, a personal digital assistant (PDA), a video camera, a notebook computer, and so on.

The multifunctional earphone may be a wireless or wired earphone that can transmit and receive data without using Bluetooth technology. The Bluetooth unit may be a wireless unit or a wired unit that can transmit and receive data without using Bluetooth technology. Data may be transmitted and received in the alcohol monitor system using wired connections or wireless connections other than a Bluetooth connection.

The setting of the reference alcohol concentration value for the multifunctional earphone may be performed by the Bluetooth alcohol-testing module, or may be performed by the transceiver module of the Bluetooth alcohol monitor host sending an instruction to the Bluetooth alcohol-testing module. The starting of the alcohol concentration-measuring program may be performed by the Bluetooth alcohol-testing module, or may be performed by the transceiver module of the Bluetooth alcohol monitor host sending an instruction to the Bluetooth alcohol-testing module.

The measured alcohol concentration value may be displayed only in the multifunctional earphone, or only in the Bluetooth alcohol monitor host, or in both the multifunctional earphone and the Bluetooth alcohol monitor host.

The reference alcohol concentration value for the multifunctional earphone may be less than the reference alcohol concentration value for the Bluetooth alcohol monitor host, or may be equal to the reference alcohol concentration value for the Bluetooth alcohol monitor host.

The notified object may be any electronic device having a communication function, such as a mobile communication terminal owned by a family member or a friend of the user of the multifunctional earphone, an ignition interlock having a communication function provided in a motor vehicle of the user of the multifunctional earphone, or a monitoring device having a communication function operated by a government or private agency that is monitoring the user's compliance with a court-ordered requirement that the user of the multifunctional earphone abstain from using alcohol.

The measured alcohol concentration value may be transmitted to the notified object through any suitable wireless communication connection, such as a GPRS (general packet radio service) connection, a GSM (global system for mobile communication) connection, a Wi-Fi (wireless fidelity) connection, a CDMA (code division multiple access) connection, a TDMA (time division multiple access) connection, an FDMA (frequency division multiple access) connection, an iDEN (integrated digital enhanced network) connection, a PDC (personal digital cellular) connection, and so on.

Accordingly, it is therefore intended that the invention not be limited to the various example embodiments disclosed herein, but that the invention include all embodiments falling within the scope of the claims and their equivalents.

What is claimed is:

1. A multifunctional earphone comprising:
an earphone body; and
an alcohol-testing module provided in the earphone body, the alcohol-testing module comprising:
an alcohol-testing unit provided with an alcohol concentration-measuring program to measure an alcohol concentration of a user of the multifunctional earphone to obtain a measured alcohol concentration value; and
a communication unit to receive the measured alcohol concentration value from the alcohol-testing unit, and analyze the measured alcohol concentration value.

2. The multifunctional earphone of claim 1, wherein the communication unit comprises:
a data processing unit to analyze the measured alcohol concentration value to obtain an analysis result;
a storage unit to store the measured alcohol concentration value and a predetermined reference alcohol concentration value set for the multifunctional earphone;
a data processing unit to analyze the measured alcohol concentration value to obtain an analysis result; and
a wireless transceiver to transmit the measured alcohol concentration value based on the analysis result obtained by the data processing unit.

3. The multifunctional earphone of claim 2, wherein the data processing unit compares the measured alcohol concentration value with the reference alcohol concentration value for the multifunctional earphone to determine whether the measured alcohol concentration value is equal to or greater than the reference alcohol concentration value for the multifunctional earphone; and
wherein the wireless transceiver transmits the measured alcohol concentration value if the data processing unit determines that the measured alcohol concentration value is equal to or greater than the reference alcohol concentration value set for the multifunctional earphone.

4. The multifunctional earphone of claim 3, further comprising an alerting unit to display the measured alcohol concentration value, and to warn the user if the data processing unit determines that the measured alcohol concentration value is equal to or greater than the predetermined reference alcohol concentration value set for the multifunctional earphone.

5. An alcohol monitor system comprising:
a multifunctional earphone comprising:
an earphone body; and
an alcohol-testing module provided in the earphone body to measure an alcohol concentration of a user of the multifunctional earphone to obtain a measured alcohol concentration value and transmit the measured alcohol concentration value; and
an alcohol monitor host comprising a transceiver module to communicate with the alcohol-testing module of the multifunctional earphone and receive the measured alcohol concentration value from the alcohol-testing module.

6. The alcohol monitor system of claim 5, wherein the alcohol-testing module comprises:
an alcohol-testing unit provided with an alcohol concentration-measuring program to measure the alcohol concentration of the user of to obtain the measured alcohol concentration value; and
a communication unit to receive the measured alcohol concentration value from the alcohol-testing unit, analyze the measured alcohol concentration value to obtain an analysis result, and transmit the measured alcohol concentration value to the alcohol monitor host based on the analysis result.

7. The alcohol monitor system of claim 6, wherein the communication unit comprises:
a storage unit to store the measured alcohol concentration value and a predetermined reference alcohol concentration value set for the multifunctional earphone;
a data processing unit to analyze the measured alcohol concentration value by comparing the measured alcohol concentration value with the predetermined reference alcohol concentration value set for the multifunctional earphone to obtain the analysis result; and
a wireless transceiver to transmit the measured alcohol concentration value to the alcohol monitor host based on the analysis result obtained by the data processing unit.

8. The alcohol monitor system of claim 7, wherein the data processing unit compares the measured alcohol concentration value with the predetermined reference alcohol concentration value for the multifunctional earphone to determine whether the measured alcohol concentration value is equal to or greater than the reference alcohol concentration value set for the multifunctional earphone; and
wherein the wireless transceiver transmits the measured alcohol concentration value to the alcohol monitor host if the data processing unit determines that the measured alcohol concentration value is equal to or greater than the predetermined reference alcohol concentration value set for the multifunctional earphone.

9. The alcohol monitor system of claim 8, wherein the alcohol-testing module further comprises an alerting unit to display the measured alcohol concentration value, and to warn the user if the data processing unit determines that the measured alcohol concentration value is equal to or greater than the reference alcohol concentration value set for the multifunctional earphone.

10. The alcohol monitor system of claim 8, wherein the alcohol monitor host further comprises a database to store a predetermined reference alcohol concentration value set for the alcohol monitor host, and to analyze the measured alcohol concentration value based on the predetermined reference alcohol concentration value set for the alcohol monitor host.

11. The alcohol monitor system of claim 10, wherein the database of the alcohol monitor host stores an information enabling the alcohol monitor host to establish a communication link with a notified object;
wherein the database is programmed to compare the measured alcohol concentration value with the reference alcohol value set for the alcohol monitor host to determine whether the measured alcohol concentration value is equal to or greater than the reference alcohol value set for the alcohol monitor host; and
wherein the transceiver module of the alcohol monitor host transmits the measured alcohol concentration value to the notified object based on the information enabling the alcohol monitor host to establish a communication link with a notified object if the database determines that the measured alcohol concentration value is equal to or greater than the reference alcohol value set for the alcohol monitor host.

12. The alcohol monitor system of claim 7, wherein the alcohol monitor host further comprises a database to store a reference alcohol concentration value for the alcohol monitor host, and to analyze the measured alcohol concentration value based on the reference alcohol concentration value set for the alcohol monitor host.

13. The alcohol monitor system of claim 12, wherein the database of the alcohol monitor host stores an information enabling the alcohol monitor host to establish a communication link with a notified object;
wherein the database compares the measured alcohol concentration value with the reference alcohol value set for the alcohol monitor host to determine whether the measured alcohol concentration value is equal to or greater than the reference alcohol value set for the alcohol monitor host; and
wherein the transceiver module of the alcohol monitor host transmits the measured alcohol concentration value to the notified object based on the information enabling the alcohol monitor host to establish a communication link with a notified object if the database determines that the measured alcohol concentration value is equal to or greater than the reference alcohol value set for the alcohol monitor host.

14. The alcohol monitor system of claim 5, wherein the alcohol monitor host is a portable electronic device having a communication function.

15. A method of measuring an alcohol concentration of a user of a multifunctional earphone, the method comprising:
measuring the alcohol concentration of the user with an alcohol concentration-measuring program provided in the multifunctional earphone to obtain a measured alcohol concentration value; and
analyzing the measured alcohol concentration value in the multifunctional earphone.

16. The method of claim 15, wherein the analyzing of the measured alcohol concentration value on the multifunctional earphone comprises comparing the measured alcohol concentration value with a predetermined reference alcohol concentration value set for the multifunctional earphone to determine whether the measured alcohol concentration value is equal to or greater than the predetermined reference alcohol concentration value set for the multifunctional earphone; and
wherein the method further comprises transmitting the measured alcohol concentration value if the measured alcohol concentration value is equal to or greater than the predetermined reference alcohol concentration value set for the multifunctional earphone.

17. The method of claim 16, wherein the transmitting of the measured alcohol concentration value comprises:
establishing a communication link between the multifunctional earphone and an alcohol monitor host;
receiving the measured alcohol concentration in the alcohol monitor host; and
analyzing the measured alcohol concentration in the alcohol monitor host.

18. The method of claim 17, wherein the analyzing of the measured alcohol concentration in the alcohol monitor host comprises comparing the measured alcohol concentration value with a predetermined reference alcohol concentration value set for the alcohol monitor host to determine whether the measured alcohol concentration value is equal to or greater than the predetermined reference alcohol concentration value set for the alcohol monitor host; and
wherein the method further comprises transmitting the measured alcohol concentration value to a notified object outside the alcohol monitor host if the measured alcohol concentration value is equal to or greater than the predetermined reference alcohol concentration value set for the alcohol monitor host.

19. The method of claim 18, wherein the predetermined reference alcohol concentration set for the multifunctional earphone is less than the predetermined reference alcohol concentration set for the alcohol monitor host.

20. The method of claim 18, wherein the notified object is a mobile communication terminal owned by a family member or a friend of the user of the multifunctional earphone, or an ignition interlock having a communication function provided in a motor vehicle of the user of the multifunctional earphone, or a monitoring device having a communication function operated by a government or private agency that is monitoring the user's compliance with a court-ordered requirement that the user of the multifunctional earphone abstain from using alcohol.
